(19) United States
(12) Patent Application Publication
(10) Pub. No.: US 2021/0350925 A1
(43) Pub. Date: Nov. 11, 2021

(54) CODE CART AND CODE CART SYSTEM

(71) Applicant: HACKENSACK MERIDIAN HEALTH, INC., Edison, NJ (US)

(72) Inventors: Winston Ramkissoon, Bridgewater, NJ (US); Sumintra Ramkissoon, Bridgewater, NJ (US)

(73) Assignee: HACKENSACK MERIDIAN HEALTH, INC., Edison, NJ (US)

(21) Appl. No.: 17/315,013

(22) Filed: May 7, 2021

Related U.S. Application Data
(60) Provisional application No. 63/022,308, filed on May 8, 2020.

(51) Int. Cl. G16H 40/67; G16H 40/20; G16H 15/00; G06K 19/07

(52) U.S. Cl. CPC G16H 40/67; G06K 19/0723; G16H 15/00; G16H 40/20

(57) ABSTRACT

A code cart is provided to securely dispense medication, equipment or medical supplies comprising disposables to a patient and provide for accounting and inventory control of the medication, equipment or medical supplies comprising disposables dispensed from the code cart. The code cart may include a processor, a memory, a housing comprising a plurality of compartments for storing the medication, equipment or medical supplies comprising disposables, an RFID tag affixed to the code cart and an RFID reader. The RFID tag uniquely identifies the code cart and the RFID reader identifies the medication, equipment or medical supplies comprising disposables stored in the code cart based on respective RFID tags affixed thereto. The process can generate information records based on read instructions executed by the RFID reader and can provide for accounting and inventory control of the medication, equipment or medical supplies comprising disposables stored in the code cart based on the generated information records.

### CROSS-REFERENCE TO RELATED APPLICATIONS

This application claims the benefit of priority to provisional application 63/022,308, filed on May 8, 2020, the contents of which is incorporated by reference in its entirety.

### FIELD OF THE INVENTION

The present disclosure relates generally to the field of medication dispensing. More specifically, the present disclosure relates to a system for securely dispensing medication, equipment, or medical supplies comprising disposables to a patient from a code cart, as well as a system for remote (cloud-based) monitoring, accounting and inventory control of the medication, equipment, or medical supplies comprising disposables dispensed from the code cart.

### BACKGROUND OF THE INVENTION

In the medication dispensing field, typical systems and methods for dispensing medication, equipment, or medical supplies comprising disposables fail to allow physicians to effectively track and monitor the usage of prescription medication by a patient to mitigate medication errors, prevent unauthorized access, and to facilitate inventory control.

Such systems and methods are problematic because of the potency and addictive nature of some prescription medication, such as opioid painkillers, and the wastefulness of expensive prescription medication (e.g., expired medication that must be discarded). Thus, a secure system and method for dispensing medication, equipment, or medical supplies comprising disposables to a patient and facilitating accounting and inventory control of such medication, equipment, or medical supplies comprising disposables is needed. Accordingly, the system of the present disclosure addresses these and other needs.

### SUMMARY OF THE INVENTION

A code cart and code cart system is provided which securely dispenses medication, equipment, or medical supplies comprising disposables to a patient and provides for accounting and inventory control of the medication, equipment, or medical supplies comprising disposables dispensed from the code cart. The code cart may include a processor, a memory, a housing comprising a plurality of compartments for storing the medication, equipment, or medical supplies comprising disposables, a locking mechanism for controlling dispensing the medication, equipment, or medical supplies comprising disposables from one of the plurality of compartments, an RFID tag affixed to the code cart and an RFID reader. The RFID tag uniquely identifies the code cart and the RFID reader identifies the medication, equipment, or medical supplies comprising disposables stored in the code cart based on respective RFID tags affixed thereto.

The processor can execute instructions to associate the code cart RFID tag with a medical provider and the respective medication, equipment, or medical supplies comprising disposables RFID tags with the code cart and the medical provider. The processor can generate a first information record based on a first read instruction executed by the RFID reader to identify the medication, equipment, or medical supplies comprising disposables stored in the code cart and can store the generated first information record in the memory. The processor can also generate a second information record based on a second read instruction executed by the RFID reader to identify remaining medication, equipment, or medical supplies comprising disposables stored in the code cart in response to the dispensing of the medication, equipment, or medical supplies comprising disposables from one of the plurality of compartments. The processor determines whether a discrepancy exists between the first information record and the second information record and updates the first information record based on the second information record when determining the discrepancy exists.

### BRIEF DESCRIPTION OF THE DRAWINGS

The foregoing features of the present disclosure will be apparent from the following Detailed Description of the Invention, taken in connection with the accompanying drawings in which:

FIG. 1 is a diagram illustrating a side view of a code cart of the system of the present disclosure;

FIG. 2 is a diagram illustrating an isometric view of the code cart of FIG. 1;

FIG. 3 is a flowchart illustrating overall processing steps carried out by the system of the present disclosure;

FIG. 4 is a flowchart illustrating step 102 of FIG. 3 in greater detail;

FIG. 5 is a flowchart illustrating step 104 of FIG. 3 in greater detail;

FIG. 6 is a flowchart illustrating step 106 of FIG. 3 in greater detail;

FIG. 7 is a flowchart illustrating step 193 of FIG. 6 in greater detail;

FIG. 8 is a flowchart illustrating step 198 of FIG. 6 in greater detail; and

FIG. 9 is a diagram illustrating hardware and software components capable of being utilized to implement the system of the present disclosure.

### DETAILED DESCRIPTION OF THE INVENTION

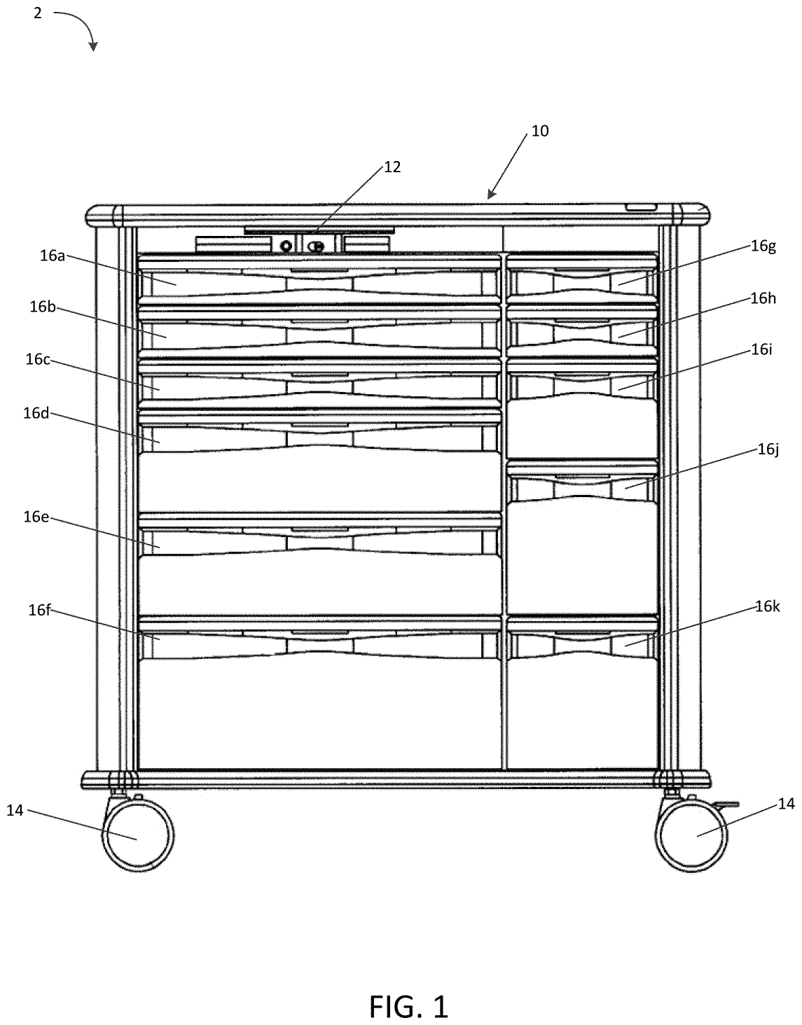

The present disclosure relates to a code cart and code cart system for securely dispensing medication, equipment, or medical supplies comprising disposables to a patient from the code cart, as well as a system for remote (cloud-based) monitoring, accounting and inventory control of the medication, equipment, or medical supplies comprising disposables dispensed from the code cart, as described in detail below in connection with FIGS. 1-9.

Turning to the drawings, FIG. 1 is a diagram 2 illustrating a side view of a code cart 10 of the system of the present disclosure. The code cart 10 includes a locking mechanism 12, wheels 14, and a plurality of drawers 16a-16k. The code cart 10 can be rectangular and the locking mechanism 12 (e.g., a break away lock) controls the opening of the drawers 16a-16k. The code cart 10 is mobile and can be transported via the wheels 14 which are mounted to a bottom of the code cart 10 and are pivotal or rotatable. The drawers 16a-16k are modular and the interior thereof can be adjusted according to a size and/or shape of medication, equipment, or medical supplies comprising disposables to be contained therein. The drawers 16a-16k and contents thereof can be removed in whole from the code cart 10 by lifting and sliding the drawers 16a-16k from the code cart 10. Alternatively, the drawers 16a-16k could each include a modular tray (not shown) positioned therein that could be removed in whole from the code cart 10. In another example, the drawers 16a-16k could include replaceable pockets such that the contents thereof could be removed in part from the code cart 10 without removing the drawers 16a-16k in whole. It should be understood that FIG. 1 is only one potential configuration, and the code cart 10 of the system of the present disclosure can have a number of different configurations. For example, in another potential configuration, the code cart 10 could include one or more drawers spanning a length of the code cart 10. In particular, a top drawer positioned directly beneath the locking mechanism 12 (e.g., merged drawers 16a and 16g) of the code cart 10 could span the length of the code cart 10 to store coded medications.

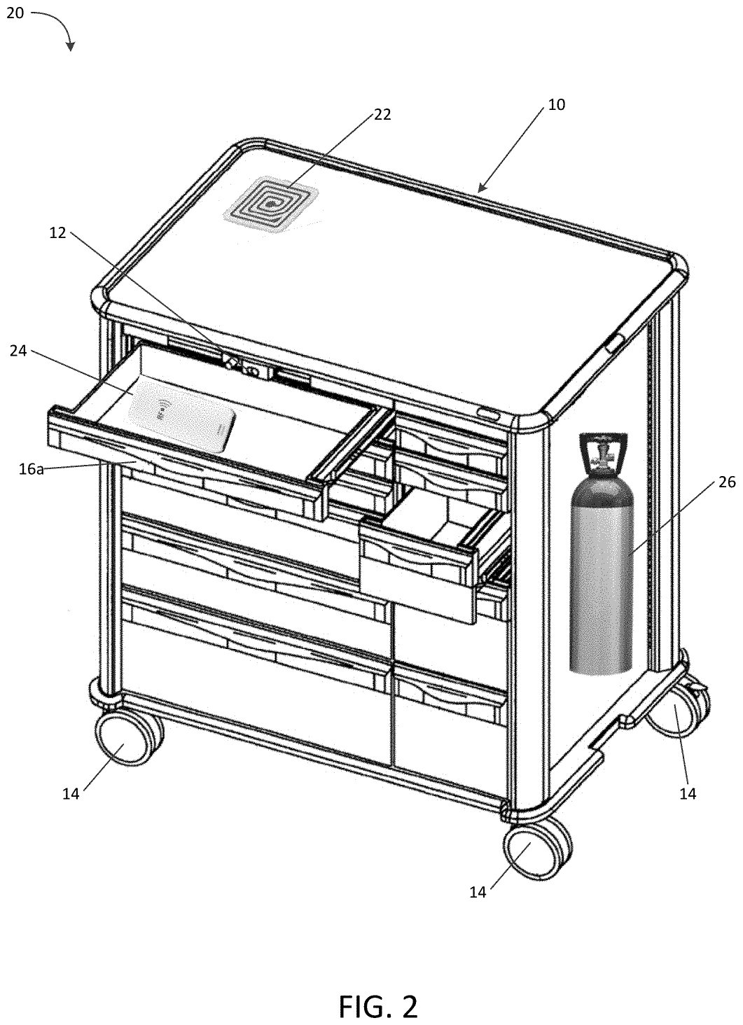

FIG. 2 is a diagram 20 illustrating an isometric view of the code cart 10 of FIG. 1. As shown in FIG. 2, the code cart 10 includes a radio frequency identification (RFID) tag 22, an RFID reader 24, and an oxygen tank 26. The RFID tag 22 can be affixed to an exterior or interior surface of the code cart 10 via an adhesive coated on a rear of the RFID tag 22. The RFID tag 22 can be passive or active and is programmable. For example, the RFID tag 22 can be programmed to be associated with the code cart 10. The RFID reader 24 can also be affixed to an exterior or interior surface of the code cart 10 via a mounting mechanism or can rest on an exterior surface (e.g., a top surface of the code cart 10) or an interior surface (e.g., within one of the plurality of drawers 16a-16k) of the code cart 10. The RFID reader 24 can read the RFID tag 22 and additional RFID tags located proximate to the code cart 10 by energizing the antenna of the RFID tag 22 and the respective antennas of additional RFID tags (e.g., the antennas of the respective RFID tags affixed to the medication, equipment, or medical supplies comprising disposables stored in the code cart 10 or affixed to an exterior of the code cart 10). The oxygen tank 26 can be affixed to an exterior of the code cart 10 via a mounting mechanism (not shown) or can be stored within one of the plurality of drawers 16a-16k. It should be understood that a variety of equipment or medical supplies comprising disposables can be affixed to an exterior of the code cart 10 via respective mounting mechanisms or can be stored within one of the plurality of drawers 16a-16k. For example, the variety of equipment or medical supplies comprising disposables could include, but are not limited to, a defibrillator, electrodes, a suction device, suction supplies, and sterile gloves of various sizes.

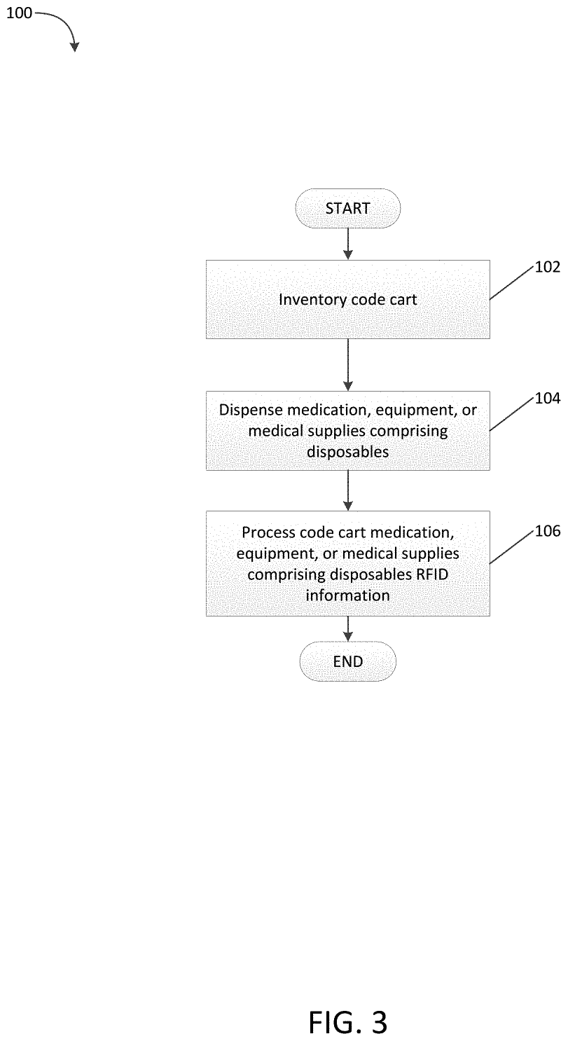

FIG. 3 is a flowchart 100 illustrating overall processing steps carried out by the system of the present disclosure. Beginning in step 102, the system executes an inventory of the code cart 10 by reading the respective RFID tags of the medication, equipment, or medical supplies comprising disposables contained therein. In step 104, the system dispenses the medication, equipment, or medical supplies comprising disposables from the code cart 10 in the event of a code (e.g., a life threatening emergency). It is noted that the medication, equipment, or medical supplies comprising disposables could be dispensed from the code cart 10 at any time deemed necessary by a medical professional (e.g., a nurse, a physician, etc.). Lastly, in step 106, the system processes code cart medication, equipment, or medical supplies comprising disposables RFID information read by the RFID reader 24. It should be understood that, based on the processing, the system provides for remote (cloud-based) monitoring, accounting and inventory control of the medication, equipment, or medical supplies comprising disposables dispensed from the code cart 10. Steps 102-106 will be described in greater detail below.

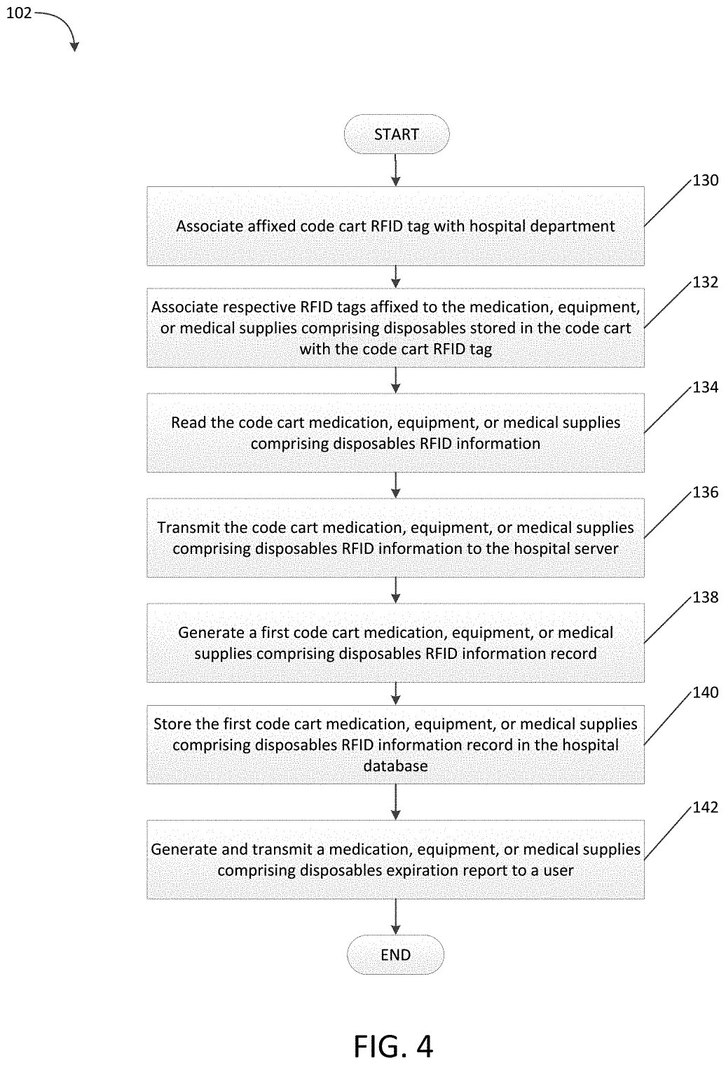

FIG. 4 is a flowchart illustrating step 102 of FIG. 3 in greater detail. In step 130, the system associates the affixed code cart RFID tag 22 with a particular medical provider (e.g., a hospital department) by programming the memory thereof. As such, the code cart 10 can be identified based on the unique Electronic Product Code (EPC) identifier associating the RFID tag 22 with the code cart 10 and by the particular hospital department with which the code cart 10 is associated. Accordingly, the contents (e.g., the medication, equipment, or medical supplies comprising disposables) of the code cart 10 can be monitored for compliance with the regulatory standards and internal hospital standards of the hospital department with which it is associated. For example, pediatrics adheres to regulatory standards different from neonatology and therefore a pediatric code cart requires medication, equipment, or medical supplies comprising disposables different from a neonatal code cart.

In step 132, the system associates respective RFID tags affixed to the medication, equipment, or medical supplies comprising disposables to be stored or stored in the code cart 10 with the code cart 10 by programming the respective memories of the RFID tags affixed to the medication, equipment, or medical supplies comprising disposables. It is noted that the RFID tags affixed to the medication, equipment, or medical supplies comprising disposables generally store information therein including, but not limited to, the manufacturer, the lot number, the unit number, the recommended dosage, and the expiration date. As such, the medication, equipment, or medical supplies comprising disposables and the RFID information thereof can be associated with the code cart 10 and the hospital department with which the code cart 10 is associated. Additionally, it should be understood that the medication, equipment, or medical supplies comprising disposables can be categorized by drawer such that the drawer categories could include, but are not limited to, common medications (e.g., alcohol swabs, Amiodarone vials, Atropine syringes, sodium bicarbonate syringes, calcium chloride syringes, sodium chloride vials, Dextrose syringes, Dopamine IV bags, Epinephrine syringes, sterile water, Lidocaine syringes, Povidone-Iodine swabstick, Vasopressin vials, etc.); pediatric medications (e.g., Atropine syringes, sodium bicarbonate syringes, saline flush syringes, sodium chloride flush syringes, etc.); intubation medical supplies (e.g., endotracheal tubes of various sizes, nasopharyngeal and oropharyngeal airways, laryngoscope handles and blades of various sizes, stylets, a bite block, tongue depressors, etc.); pediatric intubation medical supplies (e.g., laryngoscope blades, disposable Miller blades, disposable Macintosh blades, armboards of various sizes, vacutainers for blood collection, spinal needles, suction catheters of various sizes, feeding tubes, umbilical vessel catheters, disinfectants (swab sticks), pediatric IV kits, etc.); intravenous line supplies (e.g., IV start kit, angiocatheters 14 Ga and/or 16 Ga, disinfectants (Chloraprep, Betadine, povidone-iodine), Luer lock syringes of various sizes, tourniquet tubing, insyte autoguards of various sizes, vacutainers, etc.); and prepackaged medical supply kits (e.g., ECG electrodes, sterile gloves of various sizes, sutures of various sizes and materials, a cricothyroidotomy kit, a suction cath kit 14 Fr & 18 Fr, a lumbar puncture kit, etc.).

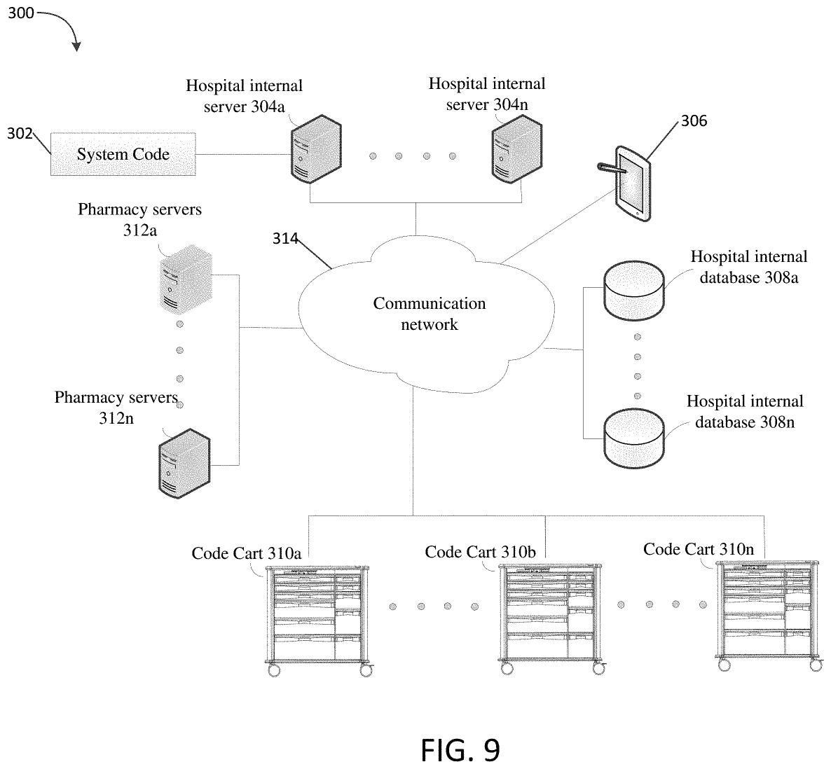

Then, in step 134, the code cart RFID reader 24 executes a read command to retrieve the information respectively associated with the medication, equipment, or medical supplies comprising disposables. In step 136, the system transmits the retrieved medication, equipment, or medical supplies comprising disposables information to a hospital server 304 (as shown in FIG. 9). The system may transmit the information over a secure network via a code cart 10 network interface in communication with the code cart RFID reader 24. The information may be provided to inventory management software being executed on the hospital server 304 via an application programming interface (API). Next, in step 138, the inventory management software generates a first code cart medication, equipment, or medical supplies comprising disposables RFID information record and in step 140, stores the RFID information record in a hospital database 308 (as shown in FIG. 9). Lastly, in step 142, the system generates and transmits a medication, equipment, or medical supplies comprising disposables expiration report to a user. The report is indicative of medication, equipment, or medical supplies comprising disposables nearing their respective expiration dates per the expiration dates stored in their respective RFID tags. The system can generate and transmit the report based on a frequency set by the user (e.g., daily, weekly, bi-weekly, monthly, etc.) or one or more fixed timed periods before the respective expiration dates of the medication, equipment, or medical supplies comprising disposables. It is noted that the report may also be transmitted to at least one of a cloud-based database, a remote operations center, and a medical provider via a network-based control system such that the database storing the report is accessible to the medical provider.

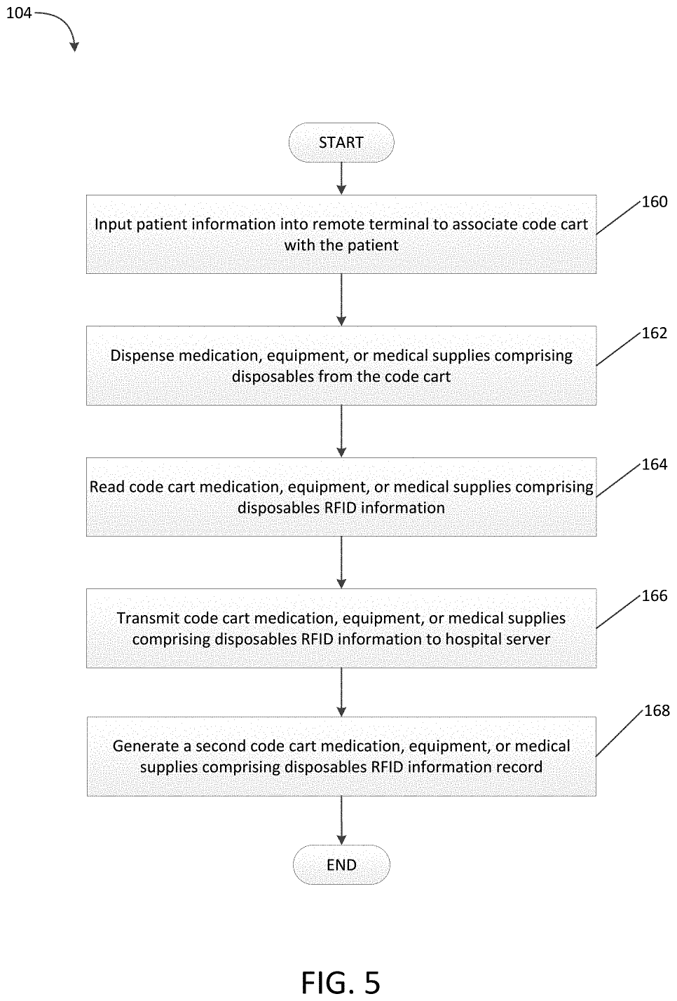

FIG. 5 is a flowchart illustrating step 104 of FIG. 3 in greater detail. Beginning in step 160, during a code, a medical professional (e.g., a nurse) tasked with documenting the code can input the patient identification information into a terminal 306 (as shown in FIG. 9) to associate the code cart 10 with the patient. The patient identification information could include, but is not limited to, first name, surname, hospital patient identification number, gender, age, sex, and social security number. The terminal 306 could include, but is not limited to, a laptop computer, a tablet, a smart telephone, a personal digital assistant (PDA) and/or a cloud-based computing platform. In step 162, the system dispenses the medication, equipment, or medical supplies comprising disposables from the code cart 10 and in step 164, the code cart RFID reader 24 executes a read command to retrieve the information respectively associated with the remaining medication, equipment, or medical supplies comprising disposables stored in the code cart 10. In step 166, the system transmits the retrieved medication, equipment, or medical supplies comprising disposables RFID information to the hospital server 304. Lastly, in step 168, the inventory management software generates a second code cart medication, equipment, or medical supplies comprising disposables RFID information record.

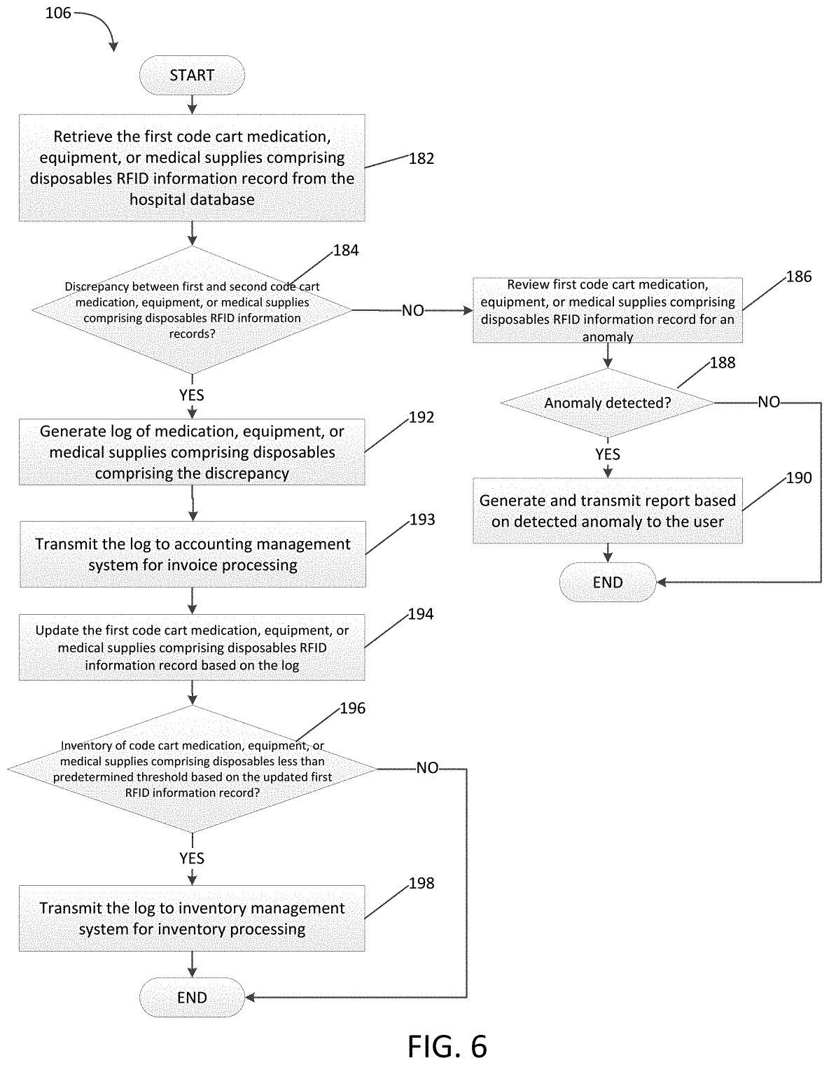

FIG. 6 is a flowchart illustrating step 106 of FIG. 3 in greater detail. Beginning in step 182, the system retrieves the first code cart medication, equipment, or medical supplies comprising disposables RFID information record from the hospital database 308. Then, in step 184, the system compares the first and second code cart medication, equipment, or medical supplies comprising disposables RFID information records to determine whether a discrepancy exists between the first and second RFID information records. The existence of a discrepancy between the first and second RFID information records confirms the dispensation of medication, equipment, or medical supplies comprising disposables from the code cart 10 during a code.

If a discrepancy does not exist, the system reviews the first code cart medication, equipment, or medical supplies comprising disposables RFID information record for at least one anomaly in step 186. In particular, the system reviews the first medication, equipment, or medical supplies comprising disposables RFID information record to determine why medication, equipment, or medical supplies comprising disposables were not dispensed from the code cart 10 during the code. For example, the system may review the respective expiration dates of the medication, equipment, or medical supplies comprising disposables stored in the code cart 10 and determine that a medication was not dispensed from the code cart because it expired.

In step 188, the system determines whether at least one anomaly is detected. If the system does not detect an anomaly, then the process ends. If the system detects an anomaly, then in step 190, the system generates and transmits a report based on the detected anomaly to the user. It is noted that the report may also be transmitted to at least one of a cloud-based database, the remote operations center, and the medical provider via a network-based control system such that the database storing the report is accessible to the medical provider. The report can include the detected anomaly in addition to information and instructions for resolving the detected anomaly. For example, if the system detects that a particular medication is expired, then the report can include the RFID information of the expired medication for its identification within and removal from the code cart 10.

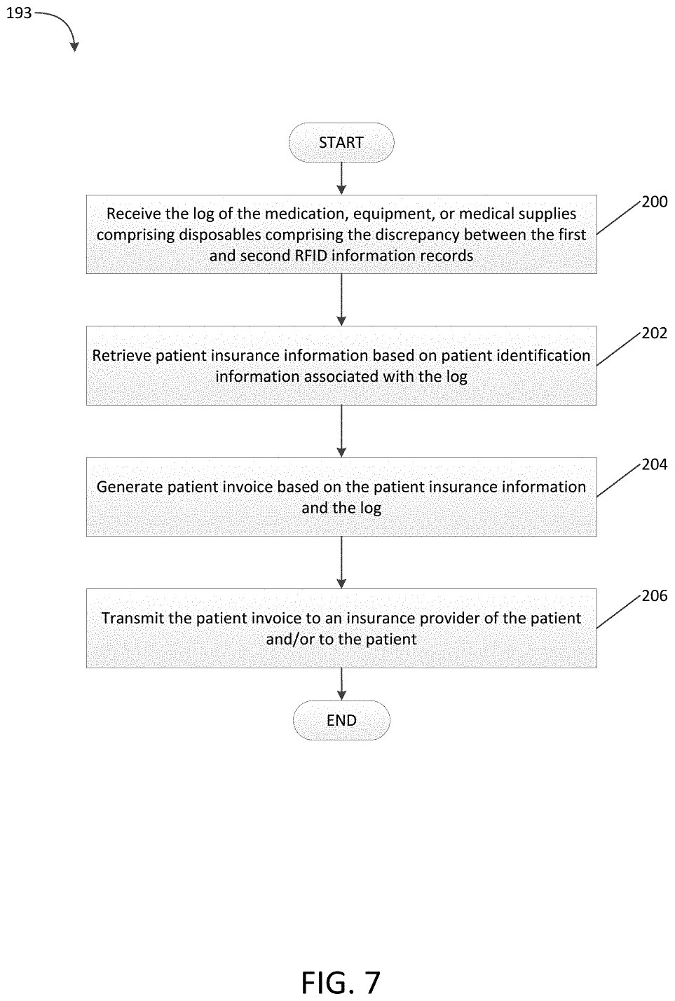

Alternatively, if the system determines a discrepancy exists between the first and second medication, equipment, or medical supplies comprising disposables RFID information records, then in step 192 the system generates a log of the medication, equipment, or medical supplies comprising disposables comprising the discrepancy. As noted above, the existence of a discrepancy between the first and second RFID information records confirms the dispensation of medication, equipment, or medical supplies comprising disposables from the code cart 10 during a code. In step 193, the system transmits the log of the medication, equipment, or medical supplies comprising disposables comprising the discrepancy to accounting management software being executed on the hospital server 304 via an API for invoice processing of the medication, equipment, or medical supplies comprising disposables dispensed from the code cart 10. Step 193 will be discussed in further detail below in relation to FIG. 7. In step 194, the system updates the first code cart medication, equipment, or medical supplies comprising disposables RFID information record based on the generated log. For example, if the log indicates that two of four syringes stored in the code cart 10 were dispensed during a code, then the system updates the first code cart medication, equipment, or medical supplies comprising disposables RFID information record to reflect that the code cart 10 only includes two syringes.

Next, in step 196, the system determines whether inventory of particular medication, equipment, or medical supplies comprising disposables stored in the code cart 10 is less than a predetermined threshold based on the updated first medication, equipment, or medical supplies comprising disposables RFID information record and regulatory and internal hospital standards. If the system determines that the inventory of the particular medication, equipment, or medical supplies comprising disposables stored in the code cart 10 is not less than the predetermined threshold then the process ends.

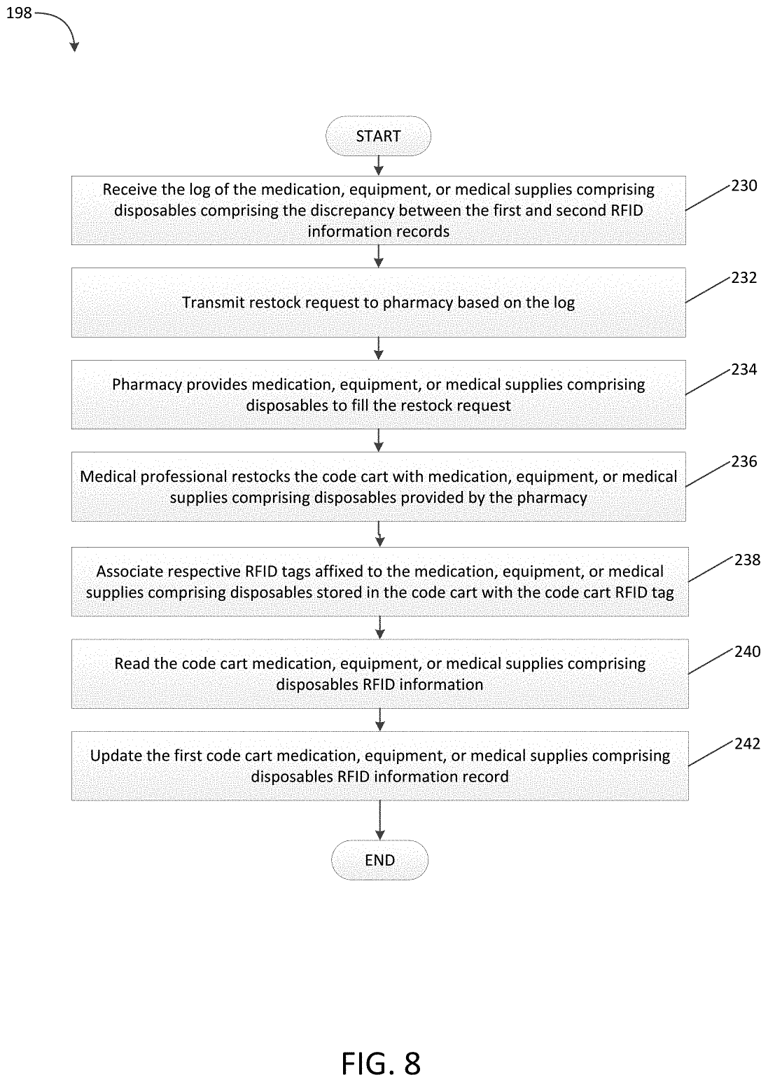

Alternatively, if the system determines that the inventory of the particular medication, equipment, or medical supplies comprising disposables stored in the code cart 10 is less than the predetermined threshold, then in step 198 the system transmits the log to the inventory management software being executed on the hospital server 304 via an API for inventory processing. Step 198 will be discussed in further detail below in relation to FIG. 8.

FIG. 7 is a flowchart illustrating step 193 of FIG. 6 in greater detail. In particular, FIG. 7 illustrates processing steps performed by the accounting management software being executed on the hospital server 304 for remote (cloud-based) invoice processing of the medication, equipment, or medical supplies comprising disposables dispensed from the code cart 10. Beginning in step 200, the accounting management software receives the log of the medication, equipment, or medical supplies comprising disposables comprising the discrepancy between the first and second medication, equipment, or medical supplies comprising disposables RFID information records. As noted above, the existence of a discrepancy between the first and second RFID information records confirms the dispensation of medication, equipment, or medical supplies comprising disposables from the code cart 10 during a code. As also noted above, during the code, a medical professional (e.g., a nurse) tasked with documenting the code inputs the patient identification information into the terminal 306 (as shown in FIG. 9) to associate the code cart 10 with the patient. Therefore, the log is indicative of medication, equipment, or medical supplies comprising disposables dispensed from the code cart 10 for the patient.

In step 202, the system retrieves patient insurance information, based on the patient identification information associated with the log, from the hospital internal database 308 (as shown in FIG. 9) or from a third party provider (e.g., an insurance provider of the patient). Then, in step 204, the system generates a patient invoice for the medication, equipment, or medical supplies comprising disposables dispensed from the code cart 10 based on the patient insurance information and the log. Lastly, in step 206, the system transmits the generated invoice to the insurance provider of the patient and/or to the patient for payment.

FIG. 8 is a flowchart illustrating step 198 of FIG. 6 in greater detail. In particular, FIG. 8 illustrates processing steps performed by the inventory management software being executed on the hospital server 304 for remote (cloud-based) inventory control of the medication, equipment, or medical supplies comprising disposables dispensed from the code cart 10. Beginning in step 230, the inventory management software receives the log of the medication, equipment, or medical supplies comprising disposables comprising the discrepancy between the first and second medication, equipment, or medical supplies comprising disposables RFID information records. As noted above, the existence of a discrepancy between the first and second RFID information records confirms the dispensation of medication, equipment, or medical supplies comprising disposables from the code cart 10 during a code. Therefore, the log is indicative of medication, equipment, or medical supplies comprising disposables dispensed from the code cart 10 during a code.

In step 232, the system transmits a restock request to a pharmacy 312 (as shown in FIG. 9) based on the log. The restock request is indicative of the medication, equipment, or medical supplies comprising disposables dispensed from the code cart 10 for which the system determined that the inventory thereof stored in the code cart 10 is less than a predetermined threshold (as shown in step 196 of FIG. 6). For example, if regulatory and internal hospital standards require that the code cart 10 store at least three syringes, then the system would transmit a restock request to the pharmacy 312 for an additional syringe to comply with the regulatory and internal hospital standards when one among the three stored syringes in the code cart 10 is dispensed during a code.

In step 234, the pharmacy 312 provides medication, equipment, or medical supplies comprising disposables to fill the restock request. It should be understood that the medication, equipment, or medical supplies comprising disposables provided by the pharmacy 312 to fill the restock request could respectively include pre-programmed RFID tags associated with the code cart 10 and affixed to the medication, equipment, or medical supplies comprising disposables. Alternatively, a medical professional (e.g., a nurse) tasked with restocking the code cart 10 could affix RFID tags to the medication, equipment, or medical supplies comprising disposables and subsequently associate the respective RFID tags with the code cart 10 RFID tag. As noted above, the respective RFID tags affixed to the medication, equipment, or medical supplies comprising disposables generally store information therein including, but not limited to, the manufacturer, the lot number, the unit number, the recommended dosage, and the expiration date. Then in step 236, the medical professional tasked with restocking the code cart 10 restocks the code cart 10 with the medication, equipment, or medical supplies comprising disposables provided by the pharmacy 312.

In step 238, the system associates the respective RFID tags affixed to the medication, equipment, or medical supplies comprising disposables provided by the pharmacy 312 and stored in the code cart 10 with the code cart 10. In particular, the system programs the respective memories of the RFID tags affixed to the medication, equipment, or medical supplies comprising disposables to be associated with the code cart 10 and the hospital department with which the code cart 10 is associated. Then, in step 240, the code cart RFID reader 24 executes a read command to retrieve the information respectively associated with the medication, equipment, or medical supplies comprising disposables. Lastly, in step 242, the system updates the first code cart medication, equipment, or medical supplies comprising disposables RFID information record based on the retrieved information.

FIG. 9 is a diagram 300 illustrating hardware and software components capable of being utilized to implement the system of the present disclosure. The system can include hospital internal servers 304a-304n having at least one processor and memory for executing the computer instructions and methods described above (which could be embodied as system code 302). The system can also include a terminal 306, a hospital internal databases 308a-308n, code carts 310a-310n, and pharmacy servers 312a-312n. The hospital internal servers 304a-304n, the terminal 306, the hospital internal databases 308a-308n, the code carts 310a-310n, and the pharmacy servers 312a-312n can communicate over a communication network 314.

Of course, the system need not be implemented on multiple devices, and indeed, the system could be implemented on a single computer system (e.g., a laptop computer, a server, etc.) without departing from the spirit or scope of the present disclosure. For example, the system could be embodied as a central processing unit (e.g., a hardware processor) coupled to the code cart 10 and in communication with pharmacy servers 312a-312n. The hardware processor could include, but is not limited to, a laptop computer, a tablet computer, a smart telephone, a server, and/or a cloud-based computing platform.

The system code 302 (i.e., non-transitory, computer-readable instructions) can be stored on a computer-readable medium and executable by a hardware processor (e.g., hospital internal servers 304a-304n) or one or more computer systems. The code 302 could include various custom-written software modules that carry out the steps/processes discussed herein. The code 302 could be programmed using any suitable programming languages including, but not limited to, C, C++, C#, Java, Python or any other suitable language. Additionally, the code 302 could be distributed across multiple computer systems in communication with each other over a communications network, and/or stored and executed on a cloud computing platform and remotely accessed by a computer system in communication with the cloud platform. The code 302 could communicate with the hospital internal databases 308a-308n, which could be stored on the same computer system as the code 302, or on one or more other computer systems in communication with the code 302.

Still further, the system could be embodied as a customized hardware component such as a field-programmable gate array (“FPGA”), application-specific integrated circuit (“ASIC”), embedded system, or other customized hardware component without departing from the spirit or scope of the present disclosure. It should be understood that FIG. 9 is only one potential configuration, and the system of the present disclosure can be implemented using a number of different configurations.

Having thus described the system and method in detail, it is to be understood that the foregoing description is not intended to limit the spirit or scope thereof. It will be understood that the embodiments of the present disclosure described herein are merely exemplary and that a person skilled in the art can make any variations and modification without departing from the spirit and scope of the disclosure. All such variations and modifications, including those discussed above, are intended to be included within the scope of the disclosure. What is desired to be protected by Letters Patent is set forth in the appended claims.

What is claimed is:

1. A code cart, comprising:
a memory;
a processor configured to generate a first information record and a second information record and in communication with the memory;
a housing comprising a plurality of compartments for storing medication, equipment or medical supplies comprising disposables, the medication respectively having radio frequency identification (RFID) tags affixed thereto;
an RFID tag for identifying the code cart; and
an RFID reader for identifying the medication, equipment or medical supplies comprising disposables based on the respective RFID tags affixed thereto, wherein
the processor
executes instructions to associate the code cart RFID tag with a medical provider and the respective medication, equipment or medical supplies comprising disposables RFID tags with the code cart and the medical provider,
executes a first read instruction via the RFID reader to identify the medication, equipment or medical supplies comprising disposables stored in the code cart,
generates the first information record based on the first read instruction,
stores the generated first information record in the memory,
executes instructions to associate the code cart RFID tag with a patient based on patient identification information,
executes a second read instruction via the RFID reader to identify remaining medication, equipment or medical supplies comprising disposables stored in the code cart in response to a dispensation of the medication, equipment or medical supplies comprising disposables from one of the plurality of compartments to the patient,
generates the second information record based on the second read instruction,
determines whether a discrepancy exists between the first information record and the second information record, the discrepancy being indicative of the medication, equipment, or medical supplies comprising disposables dispensed from one of the plurality of compartments to the patient, and
updates the first information record based on the second information record when determining the discrepancy exists.

2. The code cart of claim 1, wherein the medication, equipment or medical supplies comprising disposables include one or more of alcohol swabs, an Amiodarone vial, an Atropine syringe, a sodium bicarbonate syringe, a calcium chloride syringe, a sodium chloride vial, a Dextrose syringe, a Dopamine IV bag, an Epinephrine syringe, sterile water, a Lidocaine syringe, a Povidone-Iodine swabstick, a Vasopressin vial, a saline flush syringe, a sodium chloride flush syringe, an endotracheal tube, a nasopharyngeal and oropharyngeal airway, a laryngoscope handle and blade, a stylet, a bite block, a tongue depressor, a disposable Miller blade, a disposable Macintosh blade, an armboard, a vacutainer for blood collection, a spinal needle, a suction catheter, a feeding tube, an umbilical vessel catheter, a pediatric IV kit, an IV start kit, an angiocatheter, Chloraprep, Betadine, povidone-iodine, a Luer lock syringe, tourniquet tubing, an insyte autoguard, a vacutainer, ECG electrodes, sterile gloves, sutures, a cricothyroidotomy kit, a suction kit, a lumbar puncture kit, a defibrillator, a suction device, or an oxygen tank.

3. The code cart of claim 1, wherein the respective RFID tags affixed to the medication, equipment or medical supplies comprising disposables store one or more of an Electronic Product Code (EPC), a manufacturer, a lot number, a unit number, a recommended dosage or an expiration date.

4. The code cart of claim 3, wherein the processor generates and transmits an expiration report to a user, the report being indicative of medication, equipment, or medical supplies comprising disposables respective expiration dates based on the expiration dates stored in their respective RFID tags.

5. The code cart of claim 4, wherein the processor generates and transmits the expiration report according to a frequency set by a user or one or more fixed timed periods before respective expiration dates of the medication, equipment, or medical supplies comprising disposables.

6. The code cart of claim 1, wherein the patient identification information includes one or more of a first name, a surname, a hospital patient identification number, gender, age, sex, or a social security number.

7. The code cart of claim 1, wherein the processor
generates a log indicative of the determined discrepancy,
retrieves patient insurance information based on the patient identification information associated with the log,
generates an invoice based on the patient insurance information and the log, and
transmits the invoice to an insurance provider of the patient and/or the patient.

8. The code cart of claim 1, where in the processor
generates a log indicative of the determined discrepancy,
determines whether an inventory of the remaining medication, equipment or medical supplies comprising disposables stored in the code cart is less than a predetermined threshold based on the updated first information record, and
transmits a restock request to a medical supplier via a network-based control system based on the log when the inventory of the remaining medication, equipment or medical supplies comprising disposables stored in the code cart is less than the predetermine threshold.

9. The code cart of claim 8, wherein the processor
executes instructions to associate the respective RFID tags of medication, equipment or medical supplies comprising disposables provided by the medical supplier with the code cart and the medical provider,
executes a third read instruction via the RFID reader to identify the medication, equipment or medical supplies comprising disposables provided by the medical supplier and stored in the code cart, and
updates the first information record based on the third read instruction.

10. The code cart of claim 1, wherein the processor
detects whether the first information record includes at least one anomaly when determining the discrepancy does not exist,
generates a report when detecting the at least one anomaly, and
transmits the report to at least one of a cloud-based database, a remote operations center, and the medical provider via a network-based control system, the database storing the information and being accessible to the medical provider.

11. The code cart of claim 1, further comprising a locking mechanism for controlling dispensing the medication, equipment or medical supplies comprising disposables from one of the plurality of compartments.

12. A method for securely dispensing medication, equipment or medical supplies comprising disposables to a patient using a code cart comprising the steps of:
associating a code cart radio frequency identification (RFID) tag with a medical provider, the code cart having a housing comprising a plurality of compartments for storing medication, equipment or medical supplies comprising disposables having RFID tags affixed thereto;
associating the respective RFID tags affixed to the medication, equipment or medical supplies comprising disposables with the code cart and the medical provider;
executing a first read instruction via an RFID reader to identify the medication, equipment or medical supplies comprising disposables stored in the code cart;
generating a first information record based on the first read instruction;
storing the generated first information record in a memory;
associating the code cart RFID tag with a patient based on patient identification information;
executing a second read instruction via the RFID reader to identify remaining medication, equipment or medical supplies comprising disposables stored in the code cart in response to a dispensation of the medication, equipment or medical supplies comprising disposables from one of the plurality of compartments to the patient;
generating the second information record based on the second read instruction;
determining whether a discrepancy exists between the first information record and the second information record, the discrepancy being indicative of the medication, equipment, or medical supplies comprising disposables dispensed from one of the plurality of compartments to the patient; and
updating the first information record based on the second information record when determining the discrepancy exists.

13. The method of claim 12, further comprising the steps of:
generating and transmitting an expiration report to a user, the expiration report being indicative of medication, equipment, or medical supplies comprising disposables respective expiration dates based on expiration dates stored in their respective RFID tags, and the expiration report being generated according to a frequency set by the user or one or more fixed timed periods before respective expiration dates of the medication, equipment, or medical supplies comprising disposables.

14. The method of claim 12, further comprising the steps of:
generating a log indicative of the determined discrepancy,
retrieving patient insurance information based on the patient identification information associated with the log,
generating an invoice based on the patient insurance information and the log, and
transmitting the invoice to an insurance provider of the patient and/or the patient.

15. The method of claim 12, further comprising the steps of:
generating a log indicative of the determined discrepancy,
determining whether an inventory of the remaining medication, equipment or medical supplies comprising disposables stored in the code cart is less than a predetermined threshold based on the updated first information record, and
transmitting a restock request to a medical supplier via a network-based control system based on the log when the inventory of the remaining medication, equipment or medical supplies comprising disposables stored in the code cart is less than the predetermine threshold.

16. The method of claim 15, further comprising the steps of:
associating the respective RFID tags of medication, equipment or medical supplies comprising disposables provided by the medical supplier with the code cart and the medical provider,
executing a third read instruction via the RFID reader to identify the medication, equipment or medical supplies comprising disposables provided by the medical supplier and stored in the code cart, and
updating the first information record based on the third read instruction.

17. The method of claim 12, further comprising the steps of:
detecting whether the first information record includes at least one anomaly when determining the discrepancy does not exist,
generating a report when detecting the at least one anomaly, and
transmitting the report to at least one of a cloud-based database, a remote operations center, and the medical provider via a network-based control system, the database storing the information and being accessible to the medical provider.

18. A non-transitory computer readable medium having instructions stored thereon for securely dispensing medication, equipment or medical supplies comprising disposables to a patient using a code cart which, when executed by a processor, causes the processor to carry out the steps of:
associating a code cart radio frequency identification (RFID) tag with a medical provider, the code cart having a housing comprising a plurality of compartments for storing medication, equipment or medical supplies comprising disposables having RFID tags affixed thereto;
associating the respective RFID tags affixed to the medication, equipment or medical supplies comprising disposables with the code cart and the medical provider;
executing a first read instruction via an RFID reader to identify the medication, equipment or medical supplies comprising disposables stored in the code cart;
generating a first information record based on the first read instruction;
storing the generated first information record in a memory;
associating the code cart RFID tag with a patient based on patient identification information;
executing a second read instruction via the RFID reader to identify remaining medication, equipment or medical supplies comprising disposables stored in the code cart in response to a dispensation of the medication, equipment or medical supplies comprising disposables from one of the plurality of compartments to the patient;
generating the second information record based on the second read instruction;
determining whether a discrepancy exists between the first information record and the second information record, the discrepancy being indicative of the medication, equipment, or medical supplies comprising disposables dispensed from one of the plurality of compartments to the patient; and
updating the first information record based on the second information record when determining the discrepancy exists.

19. The non-transitory computer readable medium of claim 18, the processor further carrying out the steps of:
generating and transmitting an expiration report to a user, the expiration report being indicative of medication, equipment, or medical supplies comprising disposables respective expiration dates based on expiration dates stored in their respective RFID tags, and the expiration report being generated according to a frequency set by the user or one or more fixed timed periods before respective expiration dates of the medication, equipment, or medical supplies comprising disposables.

20. The non-transitory computer readable medium of claim 18, the processor further carrying out the steps of:
generating a log indicative of the determined discrepancy,
retrieving patient insurance information based on the patient identification information associated with the log,
generating an invoice based on the patient insurance information and the log, and
transmitting the invoice to an insurance provider of the patient and/or the patient.

21. The non-transitory computer readable medium of claim 18, the processor further carrying out the steps of:
generating a log indicative of the determined discrepancy,
determining whether an inventory of the remaining medication, equipment or medical supplies comprising disposables stored in the code cart is less than a predetermined threshold based on the updated first information record, and
transmitting a restock request to a medical supplier via a network-based control system based on the log when the inventory of the remaining medication, equipment or medical supplies comprising disposables stored in the code cart is less than the predetermine threshold.

22. The non-transitory computer readable medium of claim 21, the processor further carrying out the steps of:
associating the respective RFID tags of medication, equipment or medical supplies comprising disposables provided by the medical supplier with the code cart and the medical provider,
executing a third read instruction via the RFID reader to identify the medication, equipment or medical supplies comprising disposables provided by the medical supplier and stored in the code cart, and
updating the first information record based on the third read instruction.

23. The non-transitory computer readable medium of claim 18, the processor further carrying out the steps of:
detecting whether the first information record includes at least one anomaly when determining the discrepancy does not exist,
generating a report when detecting the at least one anomaly, and
transmitting the report to at least one of a cloud-based database, a remote operations center, and the medical provider via a network-based control system, the database storing the information and being accessible to the medical provider.
